(19) United States
(12) Patent Application Publication
(10) Pub. No.: US 2022/0003765 A1
(43) Pub. Date: Jan. 6, 2022

(54) Kit and Method

(71) Applicant: AGILE LIFE SCIENCES LTD, London (GB)

(72) Inventor: Antonio Madejon SEIZ, Madrid (ES)

(21) Appl. No.: 16/931,886

(22) Filed: Jul. 17, 2020

(30) Foreign Application Priority Data
Jul. 6, 2020 (GB) 2010372.7

(51) Int. Cl. G01N 33/569

(52) U.S. Cl. CPC G01N 33/56983

(57) ABSTRACT

A method of determining the presence or absence of an antibody in a sample, the method comprising the steps of:
(a) contacting the sample with a lysis composition comprising:
   (i) a quaternary ammonium compound; and
   (ii) a non-ionic surfactant; and
(b) contacting the sample and the lysis composition with an antibody detection device.

The present invention relates to kit and method for the detection of antibodies in a sample. In particular the invention relates to a kit and method for the detection of antibodies produced by infection with severe acute respiratory syndrome coronavirus 2 (SARS-CoV-2).

There is an ongoing world-wide pandemic of coronavirus disease 2019 (COVID-19) caused by infection with SARS-CoV-2. This pandemic has so far caused hundred of thousands of deaths. Many countries have implemented strict lockdowns to limit spread of the disease. This in turn has caused major economic damage. Emerging from lockdown relies on being able to quickly identify any new cases so infected individuals can isolate along with any close contacts. Any delay in testing or obtaining results of tests can potentially lead to more people becoming infected. It is therefore essential to provide test results for those displaying symptoms or may have come into contact with infected people as soon as possible.

A feature of COVID-19 is that many people (some estimate up to 80% of those infected) do not show any symptoms. However it is believed that these people can still transmit the disease.

Understanding of the disease is still very limited and thus as well as determining whether or not an individual is presently infected with SARS-CoV-2, it would also be very useful to be able to determine which individuals have previously been infected. Some of these may not have shown symptoms.

Current methods for testing for infection with SARS-CoV-2 involve taking a nasopharyngeal culture by inserting a swab high up the nasal passage and/or at the far back of the throat. The sample is then tested in a laboratory using a polymerase chain reaction (PCR) to identify the presence or absence of the virus.

However because the analysis must be carried out in a specialist laboratory it can take a number of days to process the results. Furthermore the swab procedure is intrusive and uncomfortable and has found to result in a number of false negatives. Thus there is an urgent need to provide a method for testing for infection with SARS-CoV-2 which is less intrusive, simpler, quicker and provides more accurate results.

The current test for previous infection with SARS-CoV-2 involves analysing a blood sample by detection of antibodies using ELISA and/or lateral flow technology. Again obtaining a sample can be uncomfortable and the results may need to be processed in a specialist laboratory.

It would be highly desirable to provide a test which could be used to more quickly, more easily and more reliably determine whether or not an individual has been infected with SARS-CoV-2.

When an individual is infected with a virus they produce antibodies to fight the infection.

Studies are still ongoing but it is known that Immunoglolsulin M (IgM), Immunoglobulin G (IgG) and Immunoglolsulin A (IgA) antibodies are produced following infection with SARS-CoV-2.

A rapid test for IgM and/or IgG and/or IgA antibodies produced by infection with SARS-CoV-2 would thus be highly beneficial. In particular it would be especially advantageous to provide a rapid test which detects SARS-CoV-2 antibodies from saliva.

Rapid tests for the detection of antibodies are generally known, and typically rely on detection of an enzyme-complexed antibody and a bound antigen using a system based on the ELISA assay.

ELISA (enzyme-linked immunosorbent assay) is a plate-based assay technique designed for detecting and quantifying peptides, proteins, antibodies, and hormones. In ELISA, an antigen must be immobilized to a solid surface and then complexed with an antibody that is linked to an enzyme. Detection is accomplished by assessing the conjugated enzyme activity via incubation with a substrate to produce a measurable product. The most crucial element of the detection strategy is a highly specific antibody-antigen interaction.

ELISAs are typically performed in 96-well (or 384-well) plates, which will passively bind antibodies and proteins. The binding and immobilization of reagents makes ELISAs simple to design and perform. Having the reactants of the ELISA immobilized to the microplate surface enables easy separation of bound from non-bound material during the assay. This ability to wash away non-specifically bound materials makes the ELISA a powerful tool for measuring specific analytes within a crude preparation. For this application the protein needs to be in the native form.

Rapid tests are usually ELISA based lateral flow devices which can detect the presence of a target substance in a liquid sample without the need for specialized and costly equipment. They are widely used in medical diagnostics for home testing, point of care testing, or laboratory use.

Lateral flow tests operate on the same principles as ELISA. In these tests a liquid sample runs along the surface of a pad with reactive molecules that show a visual positive or negative result. The pads are based on a series of capillary beds, such as pieces of porous paper, microstructured polymer, or sintered polymer. Each of these pads has the capacity to transport fluid (e.g., urine, blood, saliva) spontaneously. The sample pad acts as a sponge and holds an excess of sample fluid. Once soaked, the fluid flows to the second conjugate pad in which the manufacturer has stored freeze dried bio-active particles called conjugates in a salt-sugar matrix. The conjugate pad contains all the reagents required for an optimized chemical reaction between the target molecule (e.g., an antigen) and its chemical partner (e.g., antibody) that has been immobilized on the surface of the conjugate particle. This marks target particles as they pass through the pad and continue across to test and control lines. The test line shows a signal, often a colour change as in pregnancy tests. The control line contains affinity ligands which show whether the sample has flowed through and the bio-molecules in the conjugate pad are active. After passing these reaction zones, the fluid enters the final porous material, the wick, that simply acts as a waste container. As with ELISA proteins detected in such tests need to available in the native form.

Because proteins detected in lateral flow devices must be in native form, delays between taking a sample and the processing thereof can lead to false negatives due to antibodies in the sample unfolding and decaying. Thus means for maintaining the native form of antibodies in a sample are also highly beneficial.

It is an aim of the present invention to provide an improved testing kit and method useful for the detection of antibodies, especially antibodies indicative of infection with SARS-CoV-2, including antibodies present in saliva, which has one or more advantages over the prior art.

According to a first aspect of the present invention there is provided a method of determining the presence or absence of an antibody in a sample, the method comprising the steps of:
(a) contacting the sample with a lysis composition comprising:
   (i) a quaternary ammonium compound; and
   (ii) a non-ionic surfactant; and
(b) contacting the sample and the lysis composition with an antibody detection device.

In some embodiments the lysis composition may be prepared in situ and step (a) may involve contacting the sample with a first composition comprising component (i) and a second composition comprising component (ii). Preferably step (a) involves contacting the sample with a pre-formed lysis composition comprising component (i) and component (ii).

In some embodiments in step (b) the sample may be first contacted with the device and then contacted with the lysis composition on the device. In some embodiments step (a) is carried out first and step (b) involves contacting the composition obtained in step (a) with an antibody detection device.

According to a second aspect of the present invention there is provided a kit for detecting the presence or absence of an antibody in a sample, the kit comprising:
(1) a lysis composition comprising:
   (i) a quaternary ammonium compound; and
   (ii) a non-ionic surfactant; and
(2) an antibody detection device.

### BRIEF DESCRIPTION OF THE DRAWINGS

FIG. 1 shows lateral flow devices contacted with the mixtures described in Example 2.

FIG. 2 shows lateral flow devices contacted with the mixtures described in Example 3.

FIG. 3 shows lateral flow devices contacted with the mixtures described in Example 4.

FIG. 4 shows lateral flow devices contacted with the mixtures described in Example 5.

FIG. 5 shows lateral flow devices contacted with the mixtures described in Example 6.

FIG. 6 shows lateral flow devices contacted with the mixtures described in Example 7.

The present invention relates to a method and a kit for detecting the presence or absence of an antibody in a sample.

Preferably the present invention determines the presence or absence of an antibody indicative of infection with a disease, preferably an infectious disease.

Preferably the invention determines the presence or absence of an antibody indicative of infection with a coronavirus.

Preferably the invention determines the presence or absence of infection with SARS-CoV-2.

The invention may determine the presence or absence of IgM (SARS-CoV-2).

The invention may determine the presence or absence of IgG (SARS-CoV-2).

The invention may determine the presence or absence of IgA (SARS-CoV-2).

The invention may determine the presence or absence of IgM (SARS-CoV-2) and IgG (SARS-CoV-2).

The invention may determine the presence or absence of IgM (SARS-CoV-2) and IgA (SARS-CoV-2).

The invention may determine the presence or absence of IgA (SARS-CoV-2) and IgG (SARS-CoV-2).

The invention may determine the presence or absence of IgM (SARS-CoV-2) and IgG (SARS-CoV-2) and IgA (SARS-CoV-2).

By IgM (SARS-CoV-2) we mean to refer to the Immunoglobulin M antibody which forms following infection with SARS-CoV-2.

By IgG (SARS-CoV-2) we mean to refer to the Immunoglobulin G antibody which forms following infection with SARS-CoV-2.

By IgA (SARS-CoV-2) we mean to refer to the Immunoglobulin A antibody which forms following infection with SARS-CoV-2.

Further preferred features of the first and second aspects will now be defined.

The sample used in the method of the first aspect is preferably taken from an animal, preferably a human.

The sample is preferably a sample of bodily fluid or tissue obtained from an animal, preferably a human. Suitable bodily fluids include blood and blood components, mucus, saliva, urine, vomit, faeces, sweat, semen, vaginal secretion, tears, pus, sputum, synovial fluid and pleural fluid.

Advantageously bodily fluid or tissue samples can be used directly in the method of the present invention. It is typically not necessary to first treat or purify such samples.

The present invention can be used with samples in which antibodies are outside cells, such as blood or with samples in which antibodies are found within a cell, such as in saliva. In such embodiments the cell membrane needs to be ruptured to allow detection of antibodies.

In some preferred embodiments the sample is a blood sample or a sputum sample or a saliva sample. In some embodiments the sample is from a cheek swab, serum or blood plasma.

Advantageously the present invention can be used directly on a whole blood sample.

In some especially preferred embodiments the sample is a saliva sample.

One particular advantage of the present invention is that it can be used to detect antibodies in saliva. This is significant as it avoids the disadvantages of existing methods of testing for COVID-19 which involve an uncomfortable nasopharyngeal swab which in many cases needs to be carried out by a healthcare professional, or the taking of blood.

Antibodies found in blood are typically circulating freely and are not within a cell structure. This means that lysis of the sample is not required to detect these antibodies and blood can often be used directly in a rapid test device to detect antibodies present. However the present invention provides improved results using blood samples compared with some tests of the prior art.

For a saliva sample, it is usually necessary to lyse cells in order to release antibodies present in the sample.

Step (a) of the method of the first aspect of the present invention involves contacting the sample with a lysis composition. The lysis composition may be prepared in situ during contact with the sample. Preferably the lysis composition is fully prepared before it is contacted with the sample. The lysis composition lyses the cells present in the sample to allow release of antibodies and other material from the cell. Advantageously antibodies released maintain their native form.

The lysis composition comprises (i) a quaternary ammonium compound; and (ii) a non-ionic surfactant.

Component (i) may comprise any suitable quaternary ammonium compound.

Some preferred quaternary ammonium compounds for use herein have the formula (A):

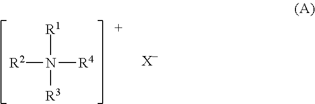

wherein each of R¹, R², R³ and R⁴ is an optionally substituted hydrocarbyl group and X⁻ is an anion.

Preferably each of R¹, R², R³ and R⁴ is an optionally substituted alkyl, alkenyl or aryl group.

Optionally substituted alkyl groups include aryl substituted alkyl groups. Substituted aryl groups include alkyl substituted aryl groups.

X⁻ may be any suitable anion. X⁻ may be selected from halide, oxyhalo, carboxylate (especially acetate), oxalate, nitrite, nitrate, sulfate, a lower alkyl sulfate, carbonate or alkyl carboxylate.

Monovalent anions are preferred. Suitable counterions include halides and oxyhalo ions for example chloride, bromide, bromite, chlorite, hypochlorite, chlorate, bromate and iodate. Preferably X⁻ is chloride or bromide. In a most preferred embodiment X⁻ is a chloride ion.

Preferably each of R¹, R², R³ and R⁴ may be optionally substituted with one or more substituents selected from halo, hydroxy, nitro, mercapto, amino, alkyl, alkoxy, aryl, sulfo, silyl, siloxy and sulfoxy. Preferred substituents are halo (especially fluoro), alkoxy and hydroxy.

Each of R¹ and R² is preferably an alkyl group having from 1 to 8 carbon atoms, most preferably 1 to 6 carbon atoms, more preferably 1 to 4 carbon atoms. R¹ and R² may suitably be selected from methyl, ethyl, propyl, butyl and isomers thereof. Preferably each of R¹ and R² is methyl or ethyl. Most preferably R¹ is methyl and R² is methyl.

Preferably R³ is an alkyl group having from 6 to 36 carbon atoms, for example from 8 to 30 carbon atoms, for example from 8 to 24 carbon atoms.

In one preferred embodiment R³ is an alkyl group having from 6 to 30 carbon atoms, preferably from 8 to 24 carbon atoms, suitably from 8 to 20 carbon atoms, for example from 10 to 18 carbon atoms and most preferably from 12 to 16 carbon atoms. The skilled person will appreciate that such groups may include a mixture of homologues.

In some embodiments R⁴ is an alkyl group having 6 to 36, preferably 6 to 30, preferably 8 to 24 or 8 to 20 carbon atoms.

In some embodiments each of R³ and R⁴ is an alkyl group having 6 to 20, preferably 8 to 16 carbon atoms, for example 10 to 12 carbon atoms. Each of R³ and R⁴ may be present as a mixture of homologues.

In some embodiments R⁴ is an alkyl aryl group, for example a benzyl group.

Suitable quaternary ammonium compounds of this type include benzyldialkyl methyl ammonium chloride and dialkyl dimethyl ammonium chloride in which the alkyl groups have 10 to 24 carbon atoms.

Some preferred quaternary ammonium compounds of this type include didecyl dimethyl ammonium chloride and dimethyl benzyl alkyl ammonium chloride in which the alkyl group contains a mixture of C₈ to C₁₆ alkyl chains.

In some embodiments R⁴ is a group of formula (B):

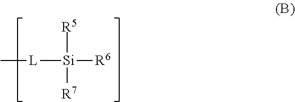

wherein L is a linking group; and each of R⁵, R⁶ and R⁷ is an optionally substituted hydrocarbyl group.

Preferred substituents which may be present in hydrocarbyl groups R⁵, R⁶ and R⁷ are halogens, in particular fluorine. In particular each of R⁵, R⁶ or R⁷ may comprise fluoroalkyl or fluoroalkoxy groups which may comprise one or more fluorine atoms.

Preferably each of R⁵, R⁶ and R⁷ is independently selected from an optionally substituted alkyl, alkenyl, aryl or alkoxy group. Preferably at least one of R⁵, R⁶ and R⁷ is an optionally substituted alkoxy group. More preferably each of R⁵, R⁶ and R⁷ is an optionally substituted alkoxy group, most preferably each is an unsubstituted alkoxy group. The alkyl group of the alkoxy group may be straight chained or branched. Preferably each of R⁵, R⁶ and R⁷ is an alkoxy group having from 1 to 20 carbon atoms, preferably from 1 to 16 carbon atoms, more preferably from 1 to 12 carbon atoms, preferably from 1 to 8 carbon atoms, suitably from 1 to 6 carbon atoms, more preferably from 1 to 4 carbon atoms.

In preferred embodiments each of R⁵, R⁶ and R⁷ is independently selected from methoxy, ethoxy, propoxy, butoxy and isomers thereof. Most preferably each of R⁵, R⁶ and R⁷ is selected from methoxy, ethoxy and isopropoxy. Preferably each of R⁵, R⁶ and R⁷ is selected from methoxy and ethoxy. Most preferably each of R⁵, R⁶ and R⁷ is methoxy. Preferably each of R⁵, R⁶ and R⁷ is the same.

L is a linking group. It may suitably be a bond or an optionally substituted alkylene, alkenylene or arylene group. Preferably L is an optionally substituted alkenylene group.

Preferably L is an unsubstituted alkylene group, more preferably an alkylene group having 1 to 12 carbon atoms, preferably 1 to 10 carbon atoms, suitably 1 to 8 carbon atoms, for example 2 to 8 carbon atoms, more preferably 2 to 6 carbon atoms, suitably 2 to 5 carbon atoms for example 2 to 4 carbon atoms. In especially preferred embodiments L is a propylene group.

In one especially preferred embodiments of the compound of formula (I), R¹, R² and R³ are each C₁ to C₄ alkoxy, L is a C₂ to C₅ alkylene group, R² and R³ are each C₁ to C₄ alkyl groups and R⁵ is a C₁₂ to C₂₄ alkyl group.

One especially preferred quaternary ammonium compound of formula (C):

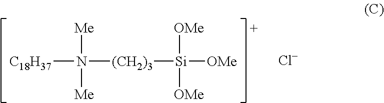

The compound shown in formula (C) is commercially available as a solution in methanol.

The skilled person will appreciate that commercial sources of such compounds may include some residual starting material and other minor impurities.

In some especially preferred embodiments the component (i) comprises a quaternary ammonium salt of formula (A) in which R¹ is C₁ to C₄ alkyl, preferably methyl; R² is C₁ to C₄ alkyl, preferably methyl; R³ is an alkyl group having 6 to 36, preferably 8 to 24 carbon atoms; and R⁴ is selected from benzyl, an alkyl group having 6 to 36, preferably 8 to 20 carbon atoms and (CH₂)₃Si(OMe)₃.

Other suitable quaternary ammonium compounds for use as component (i) include a substituted pyridinium compound, for example an alkyl or alkenyl substituted pyridinium compound. Examples include pyridinium compounds having an alkyl or alkenyl substituent of 8 to 30, preferably 10 to 20 carbon atoms. One suitable compound of this type is cetylpyridinium chloride.

In some embodiments the quaternary ammonium component (i) may be generated in situ from a precursor compound.

Some suitable precursor compounds of this type are compounds including a guanidine moiety. The composition may comprise a compound which does not contain a permanent cation but which is protonated in solution at the pH at which the composition is used. These may be referred to as precursors to quaternary ammonium compounds. Preferred are non-polymeric guanidine compounds. Examples of such compounds include chlorhexidine salts, Chlorhexidine gluconate is especially preferred.

Preferably component (i) is selected from quaternary ammonium salts of formula (A), pyridinium salts and guanidine salts.

More preferably component (i) comprises a compound of formula (A).

Most preferably component (i) comprises a compound selected from didecyl dimethyl ammonium chloride, dimethyl benzyl (C₈ to C₁₆) alkyl ammonium chloride and the compound of formula (C).

The lysis composition contacted with the sample in step (a) of the method of the present invention further comprises (ii) a non-ionic surfactant. Component (ii) may comprise any suitable non-ionic surfactant.

Preferred non-ionic surfactants are represented by the formula [A]-[B]n-H wherein A is an alkyl or alkenyl group, each B is an alkylene oxide moiety or a sugar moiety and n is at least 1.

Preferably A is an unsubstituted alkyl or alkenyl group, preferably an unsubstituted alkyl group.

It may be straight chained or may be branched. Most preferably it is straight chained. Especially preferred groups are alkyl or alkenyl groups having from 1 to 30 carbon atoms, preferably 2 to 24 carbon atoms, more preferably from 4 to 20 carbon atoms, suitably from 4 to 16 carbon atoms, preferably from 6 to 14 carbon atoms, for example from 6 to 12 carbon atoms and most preferably from 8 to 10 carbon atoms. Preferred are straight chained alkyl groups having from 6 to 12 carbon atoms. The skilled person will appreciate that such alkyl or alkenyl groups may comprise a mixture of homologues.

n is at least 1. Preferably n is from 2 to 20, preferably from 3 to 10.

In some embodiments each B is a sugar moiety, suitably a monosaccharide unit. These surfactants are known as alkyl polyglycosides. Any suitable monosaccharide unit may be included. Preferred sugar moieties include allose, altrose, glucose, mannose, gulose, idose, galactose and talose. Each B may be the same or different. Preferably each B is the same.

In some preferred embodiments at least one B is glucose. Preferably each B is glucose.

In a preferred embodiment the non-ionic surfactant (ii) is an alkyl polyglucoside (APG), preferably a monoalkyl-polyglucoside. Such compounds may be represented by the formula (D):

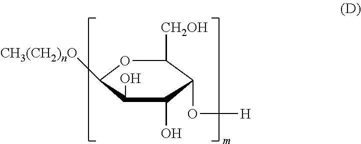

wherein n is from 5 to 12, preferably from 6 to 10, more preferably from 7 to 9 and m is from 1 to 6, preferably from 2 to 5, more preferably 3 or 4.

Other suitably non-ionic surfactants for inclusion in component (ii) are alcohol ethoxylate compounds. In such compounds each B is an alkoxy moiety, for example an ethoxy moiety or propoxy moiety. In such embodiments each B may be elected from ethoxyl, propoxy and mixtures thereof. Ethoxy is especially preferred. A is the residue of a fatty alcohol and is typically an alkyl or alkenyl group having 6 to 24, for example 8 to 18 carbon atoms. Mixtures of homologues may be present, especially if the alcohols are derived from natural sources.

Some preferred non-ionic compounds of this type have the formula CH₃(CH₂)ₘO(CH₂CH₂O)ₚH wherein m is from 5 to 20, preferably 6 to 16 and p is from 1 to 12, for example 3 to 10.

The lysis composition may be provided in any suitable form. It may consist essentially of components (i) and (ii) or it may comprise one or more further components. Suitably the lysis composition comprises one or more solvents. Preferred solvents are water and water miscible solvents.

Preferably the lysis composition is aqueous. In especially preferred embodiments water comprises at least 90 wt %, more preferably at least 95 wt % or at least 99 wt % of all solvents present in the lysis composition.

The composition may comprise a mixture of two or more quaternary ammonium compounds and/or a mixture of two or more non-ionic surfactants (ii).

The lysis composition preferably comprises from 0.00001 to 1 wt % of a quaternary ammonium compound, preferably from 0.0001 to 0.1 wt %, preferably from 0.0002 to 0.05 wt %.

In embodiments in which the lysis composition comprises more than one quaternary ammonium compound, the above amounts refer to the total of all such compounds.

The lysis composition preferably comprises from 0.00001 to 1 wt % of a non-ionic surfactant, preferably from 0.0001 wt % to 0.1 wt %, preferably from 0.0002 to 0.05 wt %.

In embodiments in which the lysis composition comprises two or more non-ionic surfactants the above amounts refer to the total of all such compounds.

The weight ratio of the quaternary ammonium compound (i) to the non-ionic surfactant (ii) present in the lysis composition is preferably from 1:50 to 20:1, preferably from 1:10 to 5:1, preferably from 1:5 to 3:1, suitably from 1:2.5 to 1.5:1.

The lysis composition preferably has a pH of from 6 to 8.

The lysis composition of the present invention may optionally comprise one or more further components, for example phosphate buffer solution (PBS), bovine serum albumin (BSA), polysorbate non-ionic surfactant, tris buffer, betaines, polyamines or other carrier molecules.

Preferably the lysis composition does not comprise a protease inhibitor.

Preferably the lysis composition consists essentially of component (i), component (ii) and water.

Step (b) of the method of the first aspect of the present invention involves contacting the sample and the lysis composition with an antibody detection device. Preferably step (b) involves contacting the composition obtained in step (a) in an antibody detection device.

Component (2) of the kit of the second aspect is an antibody detection device.

By antibody detection device we mean to refer to any device which can detect the presence or absence of one or more antibodies present in a composition.

The antibody detection device may provide a qualitative or quantitative analysis.

The device may simply identify the presence or absence of one or more antibodies in a sample.

In some embodiments the antibody detection device may determine the absolute and/or relative amounts of one or more antibodies in a sample.

Preferably there are no purification steps between step (a) and step (b). Thus in preferred embodiments the composition obtained in step (a) is contacted directly with the antibody detection device in step (b) without any additional processing steps.

In some embodiments the nature of the sample may mean that a filtration step is needed.

Preferably there are no concentration steps between step (a) and step (b).

The lysis composition provided by the invention when mixed with the sample provides a lysate composition which maintains a high concentration of soluble proteins such as antibodies. This means that no concentration steps are typically required before step (b). This also results in improved sensitivity and selectivity in step (b).

Contact of the lysis composition with a sample provides a lysate composition.

Unlike many lysis compositions of the prior art, it is not necessary in the present invention to control the pH and salt concentration of the sample and/or lysate composition in order to achieve the solubilisation of proteins such as antibodies in native form.

In preferred embodiments of the present invention it is not necessary to adjust the pH of the lysate composition.

In preferred embodiments of the present invention it is not necessary to adjust the salt concentration of the lysate composition.

According to a third aspect of the present invention there is provided the use of a lysis composition comprising (i) a quaternary ammonium compound and (ii) a non-ionic surfactant to prepare a sample for use in an antibody detection device.

Preferred features of the third aspect are as defined in relation to the first and second aspects.

The use of the third aspect of the present invention involves the use of a lysis composition to prepare a sample for use in an antibody detection device. Suitably the use involves contacting the sample with the lysis composition, preferably as defined in relation to the first aspect. Contact of the lysis composition with the sample provides a lysate composition. Preferably following contact of the lysis composition with the sample the resultant lysate composition may be directly contacted with an antibody detection device, suitably without the need for any further processing or purification steps.

Thus the third aspect of the present invention may provide the use of a lysis composition comprising (i) a quaternary ammonium compound and (ii) a non-ionic surfactant to provide a lysate composition from a sample which can be directly used in an antibody detection device.

Preferably in the lysate composition provided by the present invention antibodies maintain their 3 dimensional structure and are thus present in their native form.

The present invention provides a lysate composition in which antibodies present are soluble and stable. This is highly advantageous as it enables the lysate composition provided by step (a) to be stored without significant loss of native form and unfolding of antibodies present. This is suitably achieved without the inclusion of a protease inhibitor component.

Preferably the contact of a sample with the lysis composition of the present invention provides a lysate composition in which antibodies present are stable and maintain their native form for at least one hour, preferably at least 4 hours, suitably at least 12 hours, for example at least 24 hours or at least 48 hours.

By stable for a specific period we meant that suitably less than 20%, preferably less than 10%, more preferably less than 5% and most preferably less than 1% of the antibodies present in the lysate composition are unfolded or decayed during that period.

Preferably less than 10% of the antibodies present in the lysate composition unfold when the lysate composition is stored under ambient conditions for 12 hours.

By ambient conditions we mean to refer to atmospheric pressure and a temperature of 15 to 35° C.

The stability of the lysate composition provided by the lysis composition used in the present as it allows samples to be stored and/or transported before analysis without the risk of antibodies losing their structure or the use of complex purification steps. The lack of unfolding or decay of antibodies in the lysate composition also means that more material is available for analysis.

Preferably however the composition provided in step (a) is used immediately in step (b).

Preferably the method of the present invention provides a rapid test for the detection of antibodies in a sample.

Preferably the kit of the second aspect is a rapid detection kit.

Preferably in step (a) the lysis composition is contacted with the sample for at least 1 second, preferably at least 5 seconds.

Suitably the lysis composition is contacted with the sample for up to 10 minutes, for example up to 6 minutes.

Preferably the lysis composition is contacted with the sample in step (a) for 30 to 300 seconds. Suitably the resultant mixture is agitated to ensure mixing.

The lysis composition may be contacted with the sample by any suitable means.

In some embodiments the sample may be diluted prior to contact with the lysis composition, for example if the sample is a viscous saliva sample. The selection of a suitable diluent and amount thereof is within the competence of the person skilled in the art.

Preferably the kit of the second aspect comprises instructions for use.

Preferably the lysis composition is provided in a container. This container may comprise a specific volume of lysis composition suitable for admixture with a specific volume of the sample. Preferably an exact ratio is not necessary and there is a high degree of tolerance such that the method and kit are suitable for home use.

Preferably the kit comprises mean for collecting the sample. It may comprise means for measuring a portion of the sample. When the sample is a blood sample the kit may comprise a needle and suitable collection vial.

When the sample is a saliva sample, the kit may contain a container for collection. The kit may further comprise a syringe or microsyringe for measuring a portion of the sample.

In step (a) of the method of the first aspect of the present invention the sample is contacted with the lysis composition. Suitably the ratio of the sample composition (e.g. blood or saliva) to the lysis composition is from 10:1 to 1:1000 (sample:lysis composition). The exact ratio may depend on the nature of the sample.

In some embodiments the ratio of the sample composition to the lysis composition is from 5:1 to 1:100, suitably from 1:1 to 1:20, for example from 1:3 to 1:10, by volume.

In some embodiments the ratio of the sample composition to the lysis composition is from 10:1 to 1:2, suitably from 5:1 to 1:1, by volume.

The composition obtained in step (a) is contacted with an antibody detection device in step (b).

In some embodiments the kit of the second aspect may comprise means for measuring a portion of the composition obtained in step (a) and/or means for delivering a portion of the composition obtained in step (a) to the antibody detection device.

Such means may, for example, comprise a pipette.

In preferred embodiments the antibody detection device is a lateral flow device. Such devices are known in the art.

Suitably the lateral flow device is configured to detect one or more antibodies.

Preferably the lateral flow device includes a lateral flow immunochromatographic assay which provides a colour change when one or more specific antibodies are present in a sample. These devices are very well known and the selection of appropriate components is within the competence of the skilled person. The skilled person will also be aware of how to use such a device in order to detect antibodies.

Preferably in step (b) a portion of the composition obtained in step (a) is delivered to the sample well of a lateral flow device. However embodiments in which the sample and the lysis composition are separately delivered to the device and they are contacted on the device are also within the scope of the invention.

Preferably the lateral flow device is configured to detect IgG (SARS-CoV-2) and/or IgM (SARS-CoV-2) and/or IgA (SARS-CoV-2).

Preferably the lateral flow device is configured to detect IgG (SARS-CoV-2) and IgM (SARS-CoV-2).

The lateral flow device may be configured to detect IgG (SARS-CoV-2) and IgM (SARS-CoV-2) and IgA (SARS-CoV-2).

In some preferred embodiments in which the antibody detection device comprises a lateral flow device, the method of the first aspect may further involve contacting the antibody detection device and the sample with a carrier composition.

In such embodiments the kit of the second aspect may further comprise a carrier composition.

Suitably the carrier composition helps to transfer the sample along the lateral flow device.

Preferably the carrier composition is an aqueous composition. Preferably the carrier composition comprises one or more components selected from phosphate buffered saline, bovine serum albumin and polyalkoxylated (especially polyethoxylated) sorbitol ester non-ionic surfactants. In some embodiments the carrier composition may comprise tris buffer, betaines, polyamines or other carrier molecules.

Such carrier compositions are commonly used with lateral flow devices of this type and the skilled person would be readily be able to select appropriate components and suitable amounts thereof using their common general knowledge. Many such compositions are commercially available.

One suitable carrier composition comprises an mixture of phosphate buffered saline and polysorbate 20.

The carrier composition is suitably contacted with the device at the same time as the sample or after the sample.

In some embodiments the carrier composition may form part of the lysis composition.

In some embodiments the method may involve admixing the carrier composition with the lysis composition and then contacting the resultant mixture with the sample. Suitably in such embodiments the carrier composition and the lysis composition are mixed in a volume ratio of from 1:2 to 100:1, preferably from 1:1 to 50:1, suitably from 2:1 to 20:1.

In some embodiments the sample may be first contacted with the carrier composition and then contacted with the lysis composition before the resultant mixture is contacted with the device.

In some embodiments the sample may be first contacted with the lysis composition and then contacted with the carrier composition before the resultant mixture is contacted with the device.

In some embodiments the sample may be first contacted with the device before a mixture of the carrier composition and the lysis composition are contacted with the device.

In some embodiments the sample may be first contacted with the device, then the lysis composition is contacted with the device before the carrier composition is contacted with the device.

In some embodiments a lateral flow device may comprise a first well into which a sample is delivered and a second well into which the lysis composition and/or a carrier composition may be delivered. The configuration of devices of this type will be known to the person skilled in the art.

Any feature of any aspect of the invention may be combined with any feature of any other aspect, as appropriate.

The invention will now be further defined with reference to the following non-limiting examples.

### EXAMPLE 1

A lysis composition 1 of the invention was prepared comprising:

0.0014 wt % of di methyltetradecyl[3-(trimethoxysilyl)propyl]ammonium chloride (the compound of formula (C);

0.0014 wt % of an alkyl polyglucoside comprising 1 to 5 glucose units wherein alkyl represents a mixture of C₈ to C₁₀ alkyl groups; and water

Comparative composition 2 is a commercially available carrier composition sold along with a lateral flow device and is believed to contain polyoxyethylene sorbitol ester and bovine serum albumin.

### EXAMPLE 2

A blood sample was taken from a patient know to be positive for COVID-19.

20 μl of blood was admixed with:

A—80 μl of composition 2

B—75 μl of composition 2 plus 5 μl of water

C—75 μl of composition 2 plus 5 μl of composition 1

The mixture was shaken for 10 seconds and then 80 μl of each mixture was contacted with a lateral flow device configured to detect IgG (SARS-CoV-2) and IgM (SARS-CoV-2), according to the manufacturer's instructions.

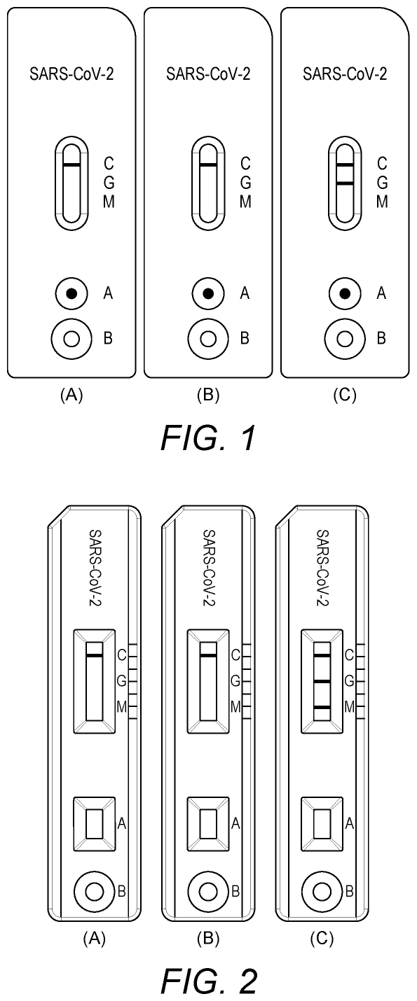

The results presented in FIG. 1 show that only device C gave a positive result for IgG (SARS-CoV-2).

### EXAMPLE 3

A saliva sample was taken from a patient know to be positive for COVID-19.

20 μl of saliva was admixed with:

A—80 μl of composition 2

B—75 μl of composition 2 plus 5 μl of water

C—75 μl of composition 2 plus 5 μl of composition 1

The mixture was shaken for 10 seconds and then 80 μl of each mixture was contacted with a lateral flow device configured to detect IgG (SARS-CoV-2) and IgM (SARS-CoV-2), according to the manufacturer's instructions.

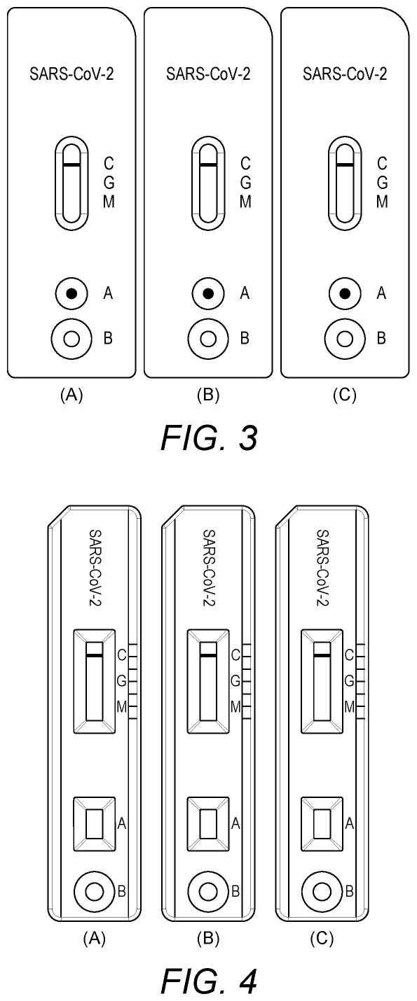

The results presented in FIG. 2 show that only device C gave a positive result for IgG (SARS-CoV-2) and IgM (SARS-CoV-2).

### EXAMPLE 4

A blood sample was taken from a patient know to be negative for COVID-19.

20 μl of blood was admixed with:

A—80 μl of composition 2

B—75 μl of composition 2 plus 5 μl of water

C—75 μl of composition 2 plus 5 μl of composition 1

The mixture was shaken for 10 seconds and then 80 μl of each mixture was contacted with a lateral flow device configured to detect IgG (SARS-CoV-2) and IgM (SARS-CoV-2), according to the manufacturer's instructions.

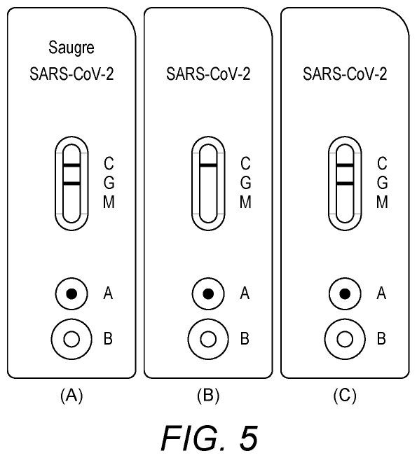

The results presented in FIG. 3 show that all devices gave a negative result.

### EXAMPLE 5

A salvia sample was taken from a patient know to be negative for COVID-19.

20 μl of saliva was admixed with:

A—80 μl of composition 2

B—75 μl of composition 2 plus 5 μl of water

C—75 μl of composition 2 plus 5 μl of composition 1

The mixture was shaken for 10 seconds and then 80 μl of each mixture was contacted with a lateral flow device configured to detect IgG (SARS-CoV-2) and IgM (SARS-CoV-2), according to the manufacturer's instructions.

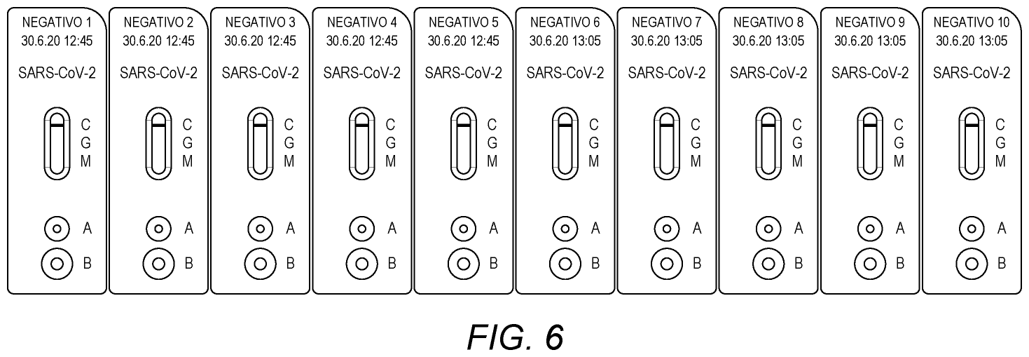

The results presented in FIG. 4 show that all devices gave a negative result.

### EXAMPLE 6

A blood sample was taken from a patient who 11 weeks earlier had tested positive for COVID-19.

20 μl of blood was added directly to the sample well of a lateral flow device, followed by:

A—80 μl of composition 2

B—75 μl of composition 2 plus 5 μl of water

C—75 μl of composition 2 plus 5 μl of composition 1

The mixture was shaken for 80 seconds and then 80 μl of the sample was contacted with a lateral flow device configured to detect IgG (SARS-CoV-2) and IgM (SARS-CoV-2), according to the manufacturer's instructions.

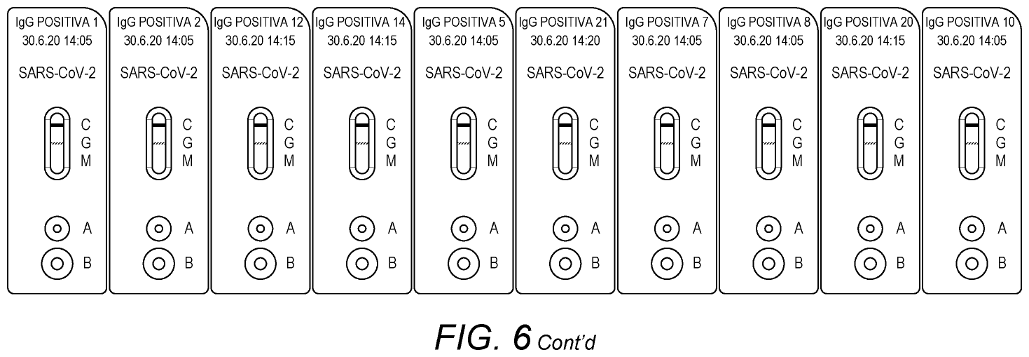

The results presented in FIG. 5 show that only devices A and C gave a positive result for IgG (SARS-CoV-2) and IgM (SARS-CoV-2).

### EXAMPLE 7

A trial was carried out on saliva samples taken from 30 patients.

All of the patients were repeatedly tested for infection with SARS-CoV-2 via a nasopharyngeal swab analysed by PCR.

Patients 1 to 10 had never been infected.

Patients 11 to 30 had previously been infected, but had since tested negative on at least 3 occasions by PCR.

All patients were asked to provide a saliva sample which was then analysed using a kit and method of the invention.

The kit contained a saliva collection device, syringes, two tubes comprising measured amounts of reagents and a lateral flow device configured to detect IgG (SARS-CoV-2) and IgM (SARS-CoV-2).

The saliva sample was collected following the standard operating procedure of Saliva Collection Aid Kit® (Salimetrics).

The collected saliva sample was inverted 5 times.

50 μl of saliva was added to a tube (tube A) comprising 155 μl of a diluent composition. The diluent composition contained 150 μL of TrisHCl (100 mM pH7.5) and 5 μL of BSA 20 mg/mL.

The resultant mixture was agitated and 50 μL was delivered to the sample well (well A) of the lateral flow device.

The other reagent tube (tube B) comprised 100 μL of a lysis/carrier composition. This was delivered to a second well (well B) of the lateral flow device.

The results were read by observing the device after 15 minutes.

Tube B contained 100 μL of a composition prepared by admixing 1860 μL of a carrier composition and 50 μL of a lysis composition.

The carrier composition contained 0.5 wt % phosphate buffered saline and 0.5 wt % polysorbate 20.

The lysis composition contained:

0.00176 wt % dimethyl benzyl alkyl ammonium chloride in which the alkyl group contains a mixture of C₈ to C₁₆ alkyl chains;

0.00352 wt % of an ethoxylated alcohol having an average of 9 to 11 atoms in an alkyl chain and 6 ethoxy residues; and

Water.

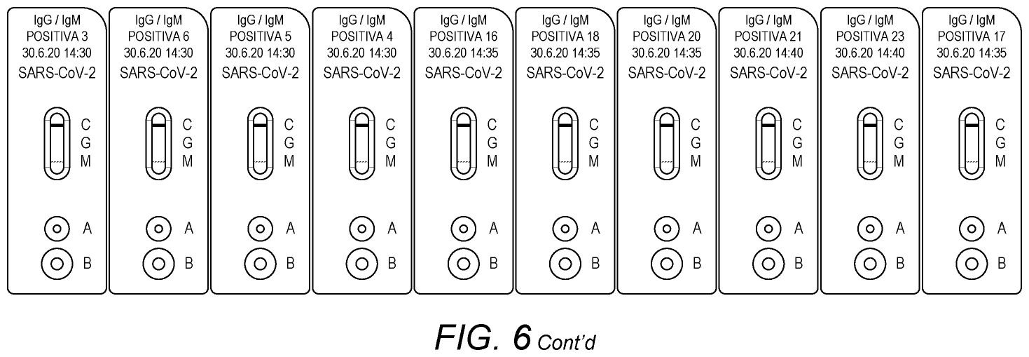

FIG. 6 shows the results for the 30 patients. Patients 1 to 10 are in the first part of FIG. 6, the other parts of FIG. 6 are patients 11 to 30.

1. A method of determining the presence or absence of an antibody in a sample, the method comprising the steps of:
(a) contacting the sample with a lysis composition comprising:
(i) a quaternary ammonium compound; and
(ii) a non-ionic surfactant; and
(b) contacting the sample and the lysis composition with an antibody detection device.

2. A method according to claim 1 wherein step (a) involves contacting the sample with a pre-formed lysis composition comprising component (i) and component (ii) and step (b) involves contacting the composition obtained in step (a) with an antibody detection device.

3. A kit for detecting the presence or absence of an antibody in a sample, the kit comprising:
(1) a lysis composition comprising:
(i) a quaternary ammonium compound; and
(ii) a non-ionic surfactant; and
(2) an antibody detection device.

4. A method according to claim 1 which detects the presence or absence of a coronavirus.

5. A method according to claim 1 which detects the presence or absence of SARS-CoV-2.

6. A method according to claim 1 wherein the sample is saliva.

7. A method according to claim 1 wherein the component (i) comprises a compound of formula (A): 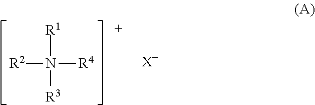
wherein each of R¹, R², R³ and R⁴ is an optionally substituted hydrocarbyl group and X⁻ is an anion.

8. A method according to claim 7 wherein X⁻ is chloride or bromide.

9. A method according to claim 7 wherein each of R¹ and R² is an alkyl group having from 1 to 4 carbon atoms.

10. A method according to claim 7 wherein R³ is an alkyl group having from 6 to 36 carbon atoms.

11. A method according to claim 7 wherein R⁴ is an alkyl group having 6 to 36 carbon atoms.

12. A method according to claim 7 wherein R⁴ is benzyl.

13. A method according to claim 7 wherein R⁴ is a group of formula (B): 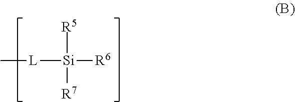
wherein L is a linking group; and each of R⁵, R⁶ and R⁷ is an optionally substituted hydrocarbyl group.

14. A method according to claim 1 wherein the non-ionic surfactant (ii) has the formula [A]-[B]ₙ—H wherein A is an alkyl or alkenyl group, each B is an alkylene oxide moiety or a sugar moiety and n is at least 1.

15. A method according to claim 14 wherein A is an unsubstituted alkyl or alkenyl group having from 4 to 20 carbon atoms.

16. A method according to claim 14 wherein n is from 2 to 10.

17. A method according to claim 14 wherein each B is glucose.

18. A method or device according to claim 14 any of claims 11 to 16 wherein each B is ethoxy.

19. (canceled)

20. A kit according to claim 3 wherein the antibody detection device comprises a lateral flow device.

21. A kit according to claim 20 wherein the lateral flow device is configured to detect IgG (SARS-CoV-2) and/or IgM (SARS-CoV-2) and/or IgA (SARS-CoV-2).

22. (canceled)
